(19) United States
(12) Patent Application Publication
(10) Pub. No.: US 2016/0174951 A1
(43) Pub. Date: Jun. 23, 2016

(54) USE OF LASER FOR EUS FNA TISSUE ACQUISITION

(71) Applicant: BOSTON SCIENTIFIC SCIMED, INC., Maple Grove, MN (US)

(72) Inventors: John HINGSTON, Framingham, MA (US); Paul MANNION, Shrewsbury, MA (US); Allison PEARLMAN, Holden, MA (US)

(21) Appl. No.: 14/971,265

(22) Filed: Dec. 16, 2015

Related U.S. Application Data
(60) Provisional application No. 62/096,336, filed on Dec. 23, 2014.

(51) Int. Cl. A61B 10/04; A61B 18/24; A61B 1/018; A61B 10/02

(52) U.S. Cl. CPC A61B 10/04; A61B 10/0266; A61B 18/24; A61B 1/018; A61B 2018/2211

(57) ABSTRACT

A needle for collecting a tissue sample includes a needle body extending longitudinally from a proximal end to a distal end and including a channel extending therethrough and a plurality of optical fibers extending along a length of the needle body and configured to pass laser energy therethrough to the distal end of the needle body to cut and collect a tissue sample within the channel.

### PRIORITY CLAIM

The present application disclosure claims priority to U.S. Provisional Patent Application Ser. No. 62/096,336 filed Dec. 23, 2014; the disclosure of which is incorporated herewith by reference.

### BACKGROUND

Needle biopsy procedures are common for the diagnosis and the staging of disease. For example, a fine needle aspiration needle may be advanced through a working channel of an endoscope to a target tissue site. Although fine needle aspiration is a highly sensitive and specific procedure, it may be difficult to acquire a suitable sample under certain clinical situations. The more cells or tissue that can be acquired, the greater the potential for a definitive diagnosis. Larger gauge needles, however, are difficult to pass along tortuous paths through anatomy to target sites and may acquire samples including more blood, making it more difficult to obtain a diagnosis.

### SUMMARY

The present disclosure is directed to a needle for collecting a tissue sample, comprising a needle body extending longitudinally from a proximal end to a distal end and including a channel extending therethrough and a plurality of optical fibers extending along a length of the needle body and configured to pass laser energy therethrough to the distal end of the needle body to cut and collect a tissue sample within the channel.

In an embodiment, the needle further comprises a sleeve mounted over the plurality of optical fibers to secure the optical fibers therealong.

In an embodiment, the plurality of optical fibers extend along one of an exterior surface of the needle body and an interior surface of the needle body.

In an embodiment, the plurality of optical fibers are embedded within a wall of the needle body.

In an embodiment, the needle body is at least one of radiopaque and echogenic.

In an embodiment, the distal end of the needle body includes a tapered tip.

In an embodiment, the needle further comprises a handle member attached to the proximal end of the needle body and including a connector engagable with a laser energy source.

In an embodiment, the connector includes a threading extending along an interior surface thereof for engaging a fiber optic line of the laser energy source.

In an embodiment, the plurality of optical fibers are equally spaced about a circumference of the needle body.

The present disclosure is also directed to a system for acquiring a tissue sample, comprising a needle extending longitudinally from a proximal end to a distal end and including a channel extending therethrough, optical fibers positioned about the needle along a length thereof such that laser energy passed through the optical fibers is delivered to the distal end of the needle and a laser energy source releasably coupleable to a proximal end of the optical fibers via a fiber optic line.

In an embodiment, the system further comprises a handle member connected to the proximal end of the needle, the handle member including a connector configured to engage a distal end of the fiber optic line.

In an embodiment, the optical fibers extend along an exterior surface of the needle.

In an embodiment, the system further comprises a sleeve mounted over the needle to secure the optical fibers thereabout, the sleeve formed of a low-friction, biocompatible material.

In an embodiment, a wavelength of the laser energy passed through the optical fiber ranges from between 0.1 micron and 11 micron.

In an embodiment, the optical fibers are equally spaced about a circumference of the needle body.

The present disclosure is also directed to a method for acquiring a tissue sample, comprising inserting a needle through a working channel of an endoscope to a target tissue within a patient body, the needle including a needle body extending longitudinally from a proximal end to a distal and a plurality of optical fibers extending along a length of the needle body and applying laser energy to the plurality of optical fibers so that laser light is delivered from the distal end of the needle to cut and collect tissue within a channel of the needle.

### BRIEF DESCRIPTION OF THE DRAWINGS

FIG. 1 shows a perspective view of a system according to an exemplary embodiment of the present disclosure;

FIG. 2 shows an enlarged perspective view of a distal portion of a needle of the system of FIG. 1;

FIG. 3 shows a longitudinal cross-sectional view of the needle of FIG. 2, along line A-A; and

FIG. 4 shows a lateral cross-sectional view of the needle of FIG. 2, along line B-

### DETAILED DESCRIPTION

The present disclosure may be further understood with reference to the following description and the appended drawings, wherein like elements are referred to with the same reference numerals. The present disclosure is related to endoscopic devices and, in particular, devices for obtaining tissue samples. Exemplary embodiments of the present disclosure describe a needle including optical fibers extending therealong for delivering laser light to a target site into which the needle is inserted to cut and collect a tissue sample within a channel of the needle. It should be noted that the terms “proximal” and “distal” as used herein, are intended to refer to a direction toward (proximal) and away from (distal) a user of the device.

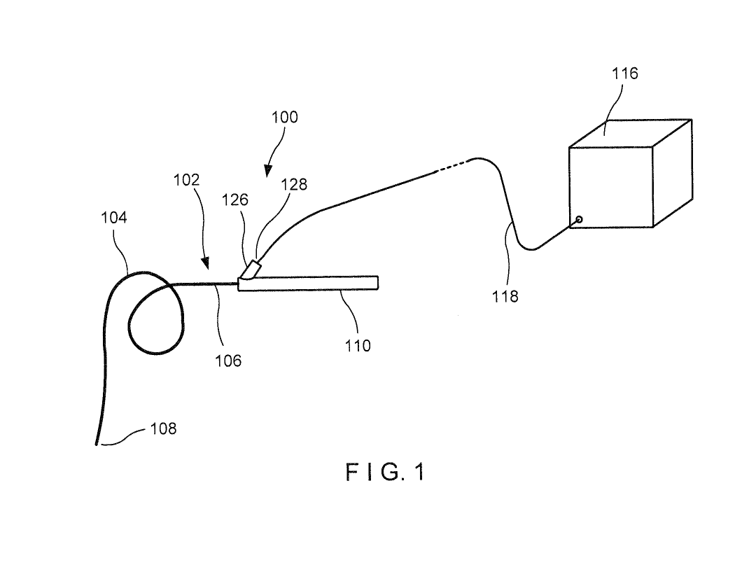

As shown in FIGS. 1-4, a system 100 according to an exemplary embodiment of the present disclosure comprises a needle 102 insertable through a working channel of an endoscope, or an endoscopic ultrasound (EUS) capable scope, to a target site within a body to collect a tissue sample. The needle 102 includes a needle body 104 extending from a proximal end 106 connected to a handle member 110 to a distal end 108 and including a channel 112 extending therethrough. Optical fibers 114 extend along the needle body 104 to deliver laser light to the distal end 108 for cutting tissue into which the distal end 108 of the needle body 104 is inserted so that a tissue sample may be collected within the channel 112. The laser light provides a smoother, cleaner cut of the tissue sample to allow for a better core sample to be collected. Blood contamination is minimized and/or eliminated from the cutting process. Conventionally, a needle is repeatedly jabbed into the target tissue to collect a tissue sample within a channel of the needle. This often produces tissue or histology with significant blood contamination. The laser light naturally cauterizes the tissue as it cuts, minimizing trauma and minimizing blood contamination from the surrounding tissue. A laser generator 116 delivers laser energy to the optical fibers 114 via a fiber optic line 118 connectable to the handle member 110.

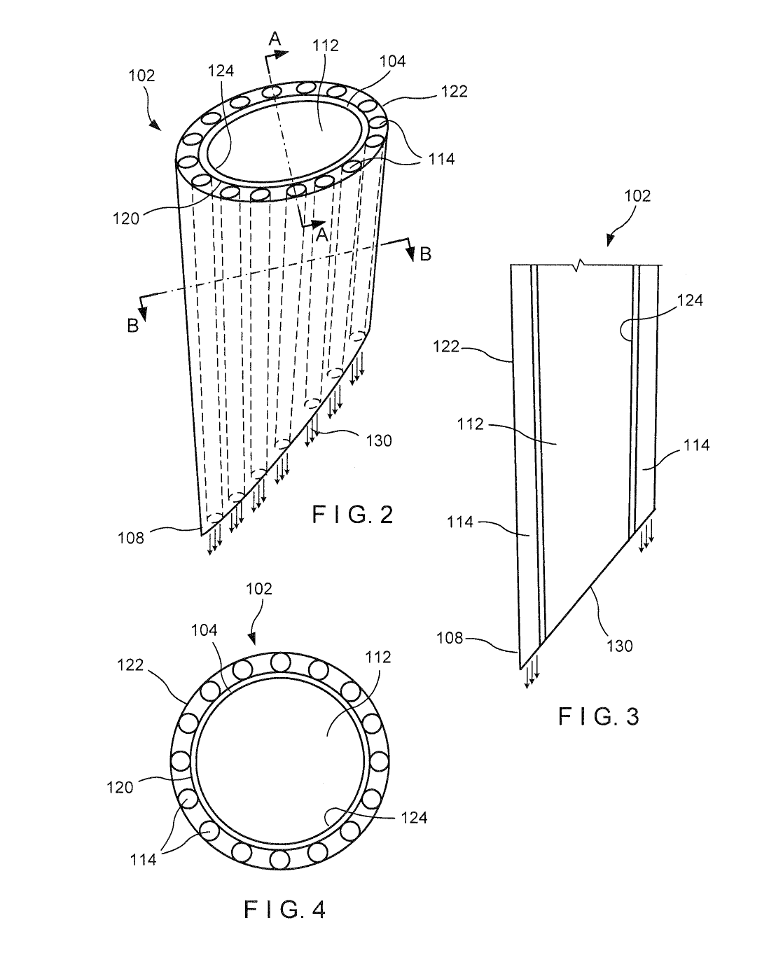

The needle body 104 extends from the proximal end 106 to the distal end 108 and is sufficiently flexible to be inserted into a living body along a tortuous path (e.g., along a path of a natural body lumen) to a target site from which a sample of target tissue is to be obtained. The needle body 104 may be formed of, for example, stainless steel or nitinol. The distal end 108 includes a sharp or tapered tip 130 to facilitate piercing target tissue. The needle body 104 or tip may be radiopaque for visualization under fluoroscopy and/or echogenic for EUS. In one exemplary embodiment, as shown in FIGS. 2-4, the optical fibers 114 are mounted along an exterior surface 120 of the needle body 104 and extend from the proximal end 106 to the distal end 108 so that each of the optical fibers 114 extends along a length of the needle body 104. The optical fibers 114 are mounted about a circumference of the needle body 104 so that laser light delivered thereby is distributed circumferentially about the channel 112. In one implementation, each of the optical fibers 114 may be equally spaced from one another about a circumference of the needle body 104. In one exemplary embodiment, the needle 102 may include between 1 and 24 optical fibers 114. A sleeve 122 may extend over the optical fibers 114 from the proximal end 106 to the distal end 108 along the length of the needle body 104 to keep the optical fibers 114 in place along the needle body 104. The sleeve 122 may be formed of any low-friction, biocompatible material such as, for example, PTFE or FEP.

A diameter of each of the optical fibers 114 may be relatively small. For example, each of the optical fibers 114 may be approximately 0.3 mm in diameter. Thus mounting the optical fibers 114 over the needle body 104 does not add much to the outer diameter of the needle body 104. Although the exemplary embodiments specifically show and describe the optical fibers 114 mounted along the exterior surface 120 of the needle body 104, the optical fibers 114 may be mounted along an interior surface 124 of the needle body 104 and fixed therealong via the sleeve 122. In another alternate embodiment, the optical fibers 114 may be embedded in a wall of the needle body 104 to extend along the length thereof.

The handle member 110 is sized and shaped to be gripped by a user of the needle 102 and is connected to the proximal end 106 of the needle body 104. The handle member 110 includes a connector 126 for engaging a distal end 128 of the fiber optic line 118. The connector 126 is sized and shaped to receive the distal end 128 of the fiber optic line 118 and is configured to pass the laser energy from the laser generator 116 to the optical fibers 114 via the fiber optic line 118. In one example, an interior of the connector 126 includes a threading configured to engage a corresponding threading along the distal end of the fiber optic line 118. The connector 126 and the distal end of the fiber optic line 118, however, may be engagable via any one of a variety of engaging mechanisms known in the art. The distal end 128 of the fiber optic line 118 may be engaged with the connector 126 to power the needle 102, when desired, and disengaged from the connector 126 upon completion of tissue acquisition.

As understood by those skilled in the art, the effect that lasers have on tissue varies both with the wavelength of the light and the duration of the pulses that the system 100 produces. Mid-infrared lasers with long wavelengths cut tissue by burning automatically cauterizing the tissue that has been cut. Shorter wavelength lasers cut via a series of micro-explosions that break molecules apart to produce precise cuts. In an exemplary embodiment, the laser generator 116 may be configured to produce laser energy having wavelengths ranging from between 0.1 micron and 11 micron and/or pulses ranging from between 100 millisecond and 10 femtosecond. The laser generator 116 may be powered on and off with a button or foot pedal actuated by a user (e.g., physician). The laser generator 116 may also be adjustable so that the user may control a degree of laser energy applied to the optical fibers 114.

According to an exemplary method using the system 100, the laser generator 116 may be connected to the needle 102 by engaging the distal end 128 of the fiber optic line 118 to the connector 126. The needle body 104 may then be inserted through a working channel of an endoscope to a target tissue site within a living body as would be understood by those skilled in the art. The tapered tip 130 at the distal end 108 of the needle body 104 is used to pierce the target tissue. The user may adjust the laser generator 116 to deliver desired laser energy (e.g., a desired intensity and duration of laser light of a selected frequency) and as controlled by a user, via, for example, a button on the generator 116 or a foot pedal connected thereto. Laser energy is passed from the laser generator 116 to the optical fibers 114 via the fiber optic line 118 such that laser light is emitted from the distal end 108 of the needle body 104. Optical fibers 114 extend about the circumference of the needle body 104 such that tissue is cut as the needle body 104 is advanced into the target tissue, collecting a tissue sample within the channel 112. As described above, the laser light provides a smooth, clean cut that minimizes or eliminates blood contamination so that a high quality core tissue sample is collected within the channel 112. Once the tissue sample has been collected within the channel 112, the laser generator 116 may be powered off and the needle 102 may be removed from the patient body. The fiber optic line 118 may be disengaged from the handle member 110 of the needle 102 so that the laser generator 116 may be used to power other needles 102.

It will be apparent to those skilled in the art that variations can be made in the structure and methodology of the present disclosure, without departing from the scope of the disclosure. Thus, it is intended that the present disclosure cover the modifications and variations of this disclosure provided that they come within the scope of the appended claims and their equivalents.

1-15. (canceled)

16. A needle for collecting a tissue sample, comprising:
a needle body extending longitudinally from a proximal end to a distal end and including a channel extending therethrough; and
a plurality of optical fibers extending along a length of the needle body and configured to pass laser energy therethrough to the distal end of the needle body to cut and collect a tissue sample within the channel.

17. The needle of claim 16, further comprising a sleeve mounted over the plurality of optical fibers to secure the optical fibers therealong.

18. The needle of claim 16, wherein the plurality of optical fibers extend along one of an exterior surface of the needle body and an interior surface of the needle body.

19. The needle of claim 16, wherein the plurality of optical fibers are embedded within a wall of the needle body.

20. The needle of claim 16, wherein the needle body is at least one of radiopaque and echogenic.

21. The needle of claim 16, wherein the distal end of the needle body includes a tapered tip.

22. The needle of claim 16, further comprising a handle member attached to the proximal end of the needle body and including a connector engagable with a laser energy source.

23. The needle of claim 16, wherein the connector includes a threading extending along an interior surface thereof for engaging a fiber optic line of the laser energy source.

24. The needle of claim 16, wherein the plurality of optical fibers are equally spaced about a circumference of the needle body.

25. A system for acquiring a tissue sample, comprising:
a needle extending longitudinally from a proximal end to a distal end and including a channel extending therethrough, optical fibers positioned about the needle along a length thereof such that laser energy passed through the optical fibers is delivered to the distal end of the needle; and
a laser energy source releasably coupleable to a proximal end of the optical fibers via a fiber optic line.

26. The system of claim 25, further comprising a handle member connected to the proximal end of the needle, the handle member including a connector configured to engage a distal end of the fiber optic line.

27. The system of claim 25, wherein the optical fibers extend along an exterior surface of the needle.

28. The system of claim 27, further comprising a sleeve mounted over the needle to secure the optical fibers thereabout, the sleeve formed of a low-friction, biocompatible material.

29. The system of claim 25, wherein a wavelength of the laser energy passed through the optical fiber ranges from between 0.1 micron and 11 micron.

30. The system of claim 25, wherein the optical fibers are equally spaced about a circumference of the needle body.

31. A method for acquiring a tissue sample, comprising:
inserting a needle through a working channel of an endoscope to a target tissue within a living body, the needle including a needle body extending longitudinally from a proximal end to a distal and a plurality of optical fibers extending along a length of the needle body; and
applying laser energy to the plurality of optical fibers so that laser light is delivered from the distal end of the needle to cut and collect tissue within a channel of the needle.

32. The method of claim 31, further comprising connecting a laser power source to the needle via an optical fiber line, wherein a distal end of the optical fiber line is inserted into a connector of a handle member attached to the proximal end of the needle body.

33. The method of claim 31, wherein the needle includes a sleeve mounted over the needle body to secure the plurality of optical fibers thereto.

34. The method of claim 31, wherein a wavelength of the laser energy applied to the plurality of optical fibers ranges from between 0.1 micron and 11 micron.

35. The method of claim 31, wherein the plurality of optical fibers are positioned about a circumference of the needle body.
